(19) United States
(12) Patent Application Publication
(10) Pub. No.: US 2019/0307401 A1
(43) Pub. Date: Oct. 10, 2019

(54) LIGHT EMISSION/RECEPTION SYSTEM AND OPTICAL BIOMETER

(71) Applicant: KONICA MINOLTA, INC., Tokyo (JP)

(72) Inventors: Kohei MIYOSHI, Tokyo (JP); Takao MISAWA, Tokyo (JP); Kenji KAWADA, Tokyo (JP)

(21) Appl. No.: 16/369,203

(22) Filed: Mar. 29, 2019

(30) Foreign Application Priority Data
Apr. 9, 2018 (JP) 2018-074710

(51) Int. Cl. A61B 5/00; A61B 5/103

(52) U.S. Cl. CPC A61B 5/6843; A61B 5/1032; A61B 5/0075

(57) ABSTRACT

A light emission and reception system for optical biometric measurement includes a light radiator and a light receiver. The light radiator radiates source light to a living body. The light receiver receives return light scattered from the living body. The light radiator and the light receiver are disposed at an end face of the system which is to be put into contact with a surface of the living body. The system measures biometric data of the living body based on intensity of the light detected by the light receiver. The light receiver comprises segments at different angular ranges around a center axis of the light radiator, and/or the light radiator comprises segments at different angular ranges around a center axis of the light receiver.

### CROSS-REFERENCE TO RELATED APPLICATION

The present invention claims priority under 35 U.S.C. § 119 to Japanese Patent Application No. 2018-074710 filed Apr. 9, 2018, the contents of which are hereby incorporated herein by reference in their entirety.

### BACKGROUND

### 1. Technological Field

The present invention relates to a light emission/reception system and an optical biometer including such a system.

### 2. Description of the Related Art

In general, the jaundice, in particular, the severe neonatal jaundice is likely to result in death. Even if one can escape from death, the jaundice may progress to the kernicterus that leaves sever aftereffects, such as cerebral palsy. Hence, the early detection of the jaundice is significantly important. In order to precisely determine the severity of the jaundice, the bilirubin levels in the sera collected from newborn infants should be measured. The blood collection from all the newborn infants, however, is difficult and is often superfluous.

A typical icteric indicator measures the intensity of the yellowish color of the bilirubin in the subcutaneous tissue of a newborn infant from the difference in optical density between the wavelength region of blue light having a central wavelength of 450 nm and the wavelength region of green light having a central wavelength of 550 nm. The tip of a probe is put into tight contact with the forehead or breast of a newborn infant to radiate light from a light source through an optical lighting fiber. The light travels through the skin and the subcutaneous tissue and then generate backscattered light. The two colors of above-mentioned wavelengths of the backscattered light are detected by a sensor. The yellowish color intensity is calculated in the ratio of the intensity of the blue light to that of the green light. The severity of the jaundice is determined from the calculated value.

The precision of the measured transdermal bilirubin level depends on the effective lengths of the optical paths in the light emission/reception systems. Thus, the systems having different effective optical paths may exhibit different results even in the case of the measurement of the transdermal bilirubin level of a single newborn infant. Hence, a means is required that reduces the unevenness of the light brightness at the light inlets of the systems to keep the center of gravity of light brightness constant.

Japanese Unexamined Patent Application Publication No. 2011-163953 discloses a light emission/reception system including a light outlet, where the unevenness of the brightness of the light from the light source is high in the circumference of the light outlet. The center of gravity of light brightness can be thereby kept stable.

Unfortunately, the invention disclosed in Japanese Unexamined Patent Application Publication No. 2011-163953 is focused only on the optical path in the light emission system and not on the optical path in the light reception system. In a typical traditional icteric indicator, the end face of the probe tilts by a specific angle from the surface of a living body of interest and is not in uniform contact with the surface, resulting in a reduction in the precision of measurement.

In detail, the following three factors adversely affects the precision of the measurement. 1. VARIATIONS IN THE OPTICAL PATH AND THE LENGTH THEREOF (i.e., shift from a predetermined optical path)

An intended design condition is that a probe P vertically stands on and is in close contact with a surface BS of the living body under a uniform pressure as illustrated in FIG. 32A. In this case, the light L emerging from a light outlet at the end face of the probe P directly enters a living body 9. The light L is scattered in and reflected from the living body 9 and is returned to a light reception face 3p at the tip of the probe P. The icteric indicator is tuned based on the data obtained by, for example, clinical evaluation to acquire a correct value under the intended condition.

As illustrated in FIG. 32B, the tilt of the probe P causes uneven pressure on the surface BS of the living body; hence, the lengths of the optical paths partially vary, resulting in fluctuation of the absorbed specific wavelengths of, for example, the blue light in the case of the icteric indicator. This affects optical characteristics, for example, the transdermal bilirubin concentrations calculated from the ratio of blue to green or TcB in the case of the icteric indicator.

As illustrated in FIG. 32C, a further tilt of the probe P causes only partial contact with the surface BS, which causes reflection of the majority of the light L at the surface BS instead of deep penetration of the light L into the living body 9. Thus, the effective lengths of the optical paths more significantly vary.

2. INTERFERENCE WITH EXTERNAL LIGHT DUE TO IMPERFECT CONTACT OF THE PROBE WITH THE SURFACE OF THE LIVING BODY

External light passes through the partial surface with which the end face of the probe P is not in contact. Typical traditional icteric indicators cancel the effects of the external light (and the fluctuation of the ambient light) through the subtracting operation of dark counts indicating the times where no light is radiated between the cycles of the light radiation. Since the acquisition of the dark counts inevitably shifts from the acquisition of the light radiation counts, components that cannot be completely cancelled affect the observed results.

Even in the case of a slight tilt of the probe P as illustrated in FIG. 32B, uneven contact with the surface BS is likely to cause a variation in incident external light from a significantly shallow depth of the living body 9.

### 3. VARIATION IN ISCHEMIC LEVEL

Traditional icteric indicators may include a switch operable in cooperation with pressing of the probe P onto the surface BS. This switch can restrict the subcutaneous blood flow to reduce the optical effects of, for example, hemoglobins in the blood (the spring force of the switch optimizes the ischemic levels). Unfortunately, the tilting probe P causes the contact area to decrease and the pressure to increase even under a constant load. The uneven pressure fluctuates the ischemic levels.

The factors described above are summarized in Table 1.

TABLE 1

| | Completely vertical in | | |
|---|---|---|---|
| | close contact with surface | Tilted, but in contact | Tilted, incomplete contact |
| | of living body (FIG. 32A) | with surface of living | with surface of living body |
| | (Ideal state) | body (FIG. 32B) | (FIG. 32C) |
| 1. Variations in | Acceptable (a | Unacceptable (reduced | Unacceptable (Incomplete |
| optical path and | predetermined | length under high | contact of light reception face |
| length thereof | length) | pressure and increased | precludes reception of return |
| | | length under low | light. Incomplete contact of |
| | | pressure) | the light radiating face |
| | | | precludes light incident to |
| | | | living body) |
| 2. Interference with | Acceptable (no | Undesirable (Some | Unacceptable (Very |
| external light due to | interference with | interference with | significant interference with |
| imperfect contact | external light) | external light at | external light at incomplete |
| | | incomplete contact | contact area) |
| | | area) | |
| 3. Variation in | Acceptable (within | Unacceptable (increased | Unacceptable (Pressure |
| ischemic level | predetermined | ischemic level under high | concentrated to contact area, |
| | ischemic level) | pressure and decreased | highly increased ischemic |
| | | ischemic level under low | level) |
| | | pressure) | |
| Overall evaluation | Excellent | Medium | Unacceptable |

### SUMMARY

An object of the present invention is to provide a light emission/reception system and an optical biometer using such a system that can detect the uniformity of the contact of the end face of a probe with a surface of a living body and thereby improve the precision or reproducibility and accuracy of measurement affected by the uneven contact.

To achieve at least one of the abovementioned objects, according to a first aspect of the present invention, a light emission and reception system for optical biometric measurement includes:

a light radiator that radiates source light to a living body; and

a light receiver that receives return light scattered from the living body,

wherein

the light radiator and the light receiver are disposed at an end face of the system which is to be put into contact with a surface of the living body during the optical biometric measurement,

the system measures biometric data of the living body based on intensity of the light detected by the light receiver, and

(i) the light receiver comprises segments which respectively detect an intensity of the return light and which are at different angular ranges around a center axis of the light radiator, and/or (ii) the light radiator comprises segments which respectively radiate the source light and which are at different angular ranges around a center axis of the light receiver.

According to a second aspect of the present invention, an optical biometer includes:

the light emission and reception system; and

a control/arithmetic processor that controls radiation of the source light through the light radiator and calculates an observed result from the return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

if a difference among the light intensities detected for the segments of the light radiator falls within a predetermined threshold, the control/arithmetic processor calculates the observed result.

According to a third aspect of the present invention, an optical biometer includes:

the light emission and reception system; and

a control/arithmetic processor that controls radiation of the source light through the light radiator and calculates an observed result from a return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

the control/arithmetic processor controls preliminary radiation of the source light and, if a difference among the light intensities detected for the segments of the light radiator during the preliminary radiation falls within a predetermined threshold, the control/arithmetic processor controls true radiation of the source light and calculates the observed result from true light intensities detected in the light receiver.

According to a fourth aspect of the present invention, an optical biometer includes:

the light emission and reception system; and

a control/arithmetic processor that controls radiation of the source light through the light radiator and calculates an observed result from a return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

the control/arithmetic processor controls the light radiator not to radiate and, if a difference among light intensities detected for the segments of the light radiator during no radiation falls within the predetermined threshold, the control/arithmetic processor controls true radiation of the source light and calculates the observed result from the true light intensities detected in the light receiver.

According to a fifth aspect of the present invention, an optical biometer includes:

the light emission and reception system; and

a control/arithmetic processor that controls radiation of the source light through the light radiator and calculates an observed result from a return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

if a difference among the light intensities detected for the segments of the light radiator exceeds a predetermined threshold, the control/arithmetic processor calculates the observed result from the light intensities detected for some of the segments of the light radiator.

According to a sixth aspect of the present invention, an optical biometer includes:

the light emission and reception system; and

a control/arithmetic processor that controls radiation of the source light through the light radiator and calculates an observed result from a return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

the control/arithmetic processor (i) controls predetermined cycles of sequential radiation of the source light for each of the segments of the light radiator, (ii) calculates a difference among the light intensities for the segments for each cycle of radiation, and (iii) calculates the observed result from relatively even light intensities detected in the light receiver.

According to a seventh aspect of the present invention, an optical biometer includes:

the light emission and reception system; and

a control/arithmetic processor that controls radiation of the source light through the light radiator and calculates an observed result from a return light detected in the light receiver,

wherein

if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and

in order to calculate the observed result from the light intensities for the segments of the light radiator, the control/arithmetic processor adds an offset according to the difference among the light intensities for the segments.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1A is a schematic perspective view of a configuration of an optical biometer according to an embodiment of the present invention.

FIG. 1B is a cross-sectional view of two optical paths for measurement of optical information on a living body.

FIG. 2 is a schematic view of the end face of a probe according to the embodiment of the invention.

FIG. 3 is a plan view of a configuration of a light outlet of a backward optical fiber bundle according to the embodiment of the invention.

FIG. 4A is another plan view of the configuration of the light outlet of the backward optical fiber bundle according to the embodiment of the invention.

FIG. 4B is a side view of the configuration of the light outlet of the backward optical fiber bundle according to the embodiment of the invention.

FIG. 5A is a plan view of a configuration of a light outlet of a backward optical fiber bundle according to another embodiment of the invention.

FIG. 5B is a side view of the configuration of the light outlet of the backward optical fiber bundle according to the embodiment of the invention.

FIG. 6 is another side view of the configuration of the light outlet of the backward optical fiber bundle according to the embodiment of the invention.

FIG. 7 is a plan view of a configuration of a light outlet of a backward optical fiber bundle according to another embodiment of the invention.

FIG. 8A is a plan view of a configuration of a light outlet of a backward optical fiber bundle according to another embodiment of the invention.

FIG. 8B is a side view of the configuration of the light outlet of the backward optical fiber bundle according to the embodiment of the invention.

FIG. 9 is a schematic view of the end face of a probe according to another embodiment of the invention.

FIG. 10 is a schematic view of the end face of a probe according to another embodiment of the invention.

FIG. 11 is a schematic view of the end face of a probe according to another embodiment of the invention.

FIG. 12 is a schematic view of the end face of a probe according to another embodiment of the invention.

FIG. 13 is a schematic view of the end face of a probe according to another embodiment of the invention.

FIG. 14 is a schematic view of the end face of a probe according to another embodiment of the invention.

FIG. 15 is a schematic view of the end face of a probe according to another embodiment of the invention.

FIG. 16 is a schematic view of the end face of a probe according to another embodiment of the invention.

FIG. 17 is a schematic view of the end face of a probe according to another embodiment of the invention.

FIG. 18A is a perspective view of an exemplary appearance of an optical biometer according to an embodiment of the invention.

FIG. 18B is a schematic view of a top panel display of the optical biometer according to the embodiment of the invention.

FIG. 18C is a schematic view of a side display of the optical biometer according to the embodiment of the invention.

FIG. 19A is a schematic view of the end face of a probe according to an embodiment of the invention.

FIG. 19B1 is a plan view of a configuration of a light outlet of an exemplary backward optical fiber bundle according to an embodiment of the invention.

FIG. 19B2 is a side view of the configuration of the light outlet of the exemplary backward optical fiber bundle according to the embodiment of the invention.

FIG. 19C1 is a plan view of a configuration of a light outlet of an exemplary backward optical fiber bundle according to another embodiment of the invention.

FIG. 19C2 is a side view of the configuration of the light outlet of the exemplary backward optical fiber bundle according to the embodiment of the invention.

FIG. 19D1 is a plan view of a configuration of a light outlet of an exemplary backward optical fiber bundle according to another embodiment of the invention.

FIG. 19D2 is a side view of the configuration of the light outlet of the exemplary backward optical fiber bundle according to the embodiment of the invention.

FIG. 20A is a schematic view of the end face of a probe according to an embodiment of the invention.

FIG. 20B1 is a plan view of a configuration of a light inlet of a forward optical fiber bundle according to the embodiment of the invention.

FIG. 20B2 is a side view of the configuration of the light inlet of the forward optical fiber bundle according to the embodiment of the invention.

FIG. 21A is a schematic view of the end face of a probe according to an embodiment of the invention.

FIG. 21B1 is a plan view of a light outlet of a backward optical fiber bundle according to the embodiment of the invention.

FIG. 21B2 is a side view of the light outlet of the backward optical fiber bundle according to the embodiment of the invention.

FIG. 21C1 is a plan view of a configuration of a light inlet of the forward optical fiber bundle according to the embodiment of the invention.

FIG. 21C2 is a side view of the configuration of the light inlet of the forward optical fiber bundle according to the embodiment of the invention.

FIG. 22A is a schematic view of the end face of an exemplary probe according to an embodiment of the invention.

FIG. 22B is a schematic view of the end face of an exemplary probe according to another embodiment of the invention.

FIG. 23A is a schematic vertical cross-sectional view of a probe including a plurality of light receiver segments and a single light radiator.

FIG. 23B is a schematic vertical cross-sectional view of a probe including two light receiver segments and two light radiator segments.

FIG. 24A is a schematic vertical cross-sectional view of a probe according to an embodiment of the invention.

FIG. 24B is a schematic vertical cross-sectional view of the probe according to the embodiment of the invention.

FIG. 25A is a schematic side view of a probe according to another embodiment of the invention.

FIG. 25B is a schematic side view of a probe according to the embodiment of the invention.

FIG. 26A is a vertical cross-sectional view of a probe according to another embodiment of the invention.

FIG. 26B is a vertical cross-sectional view of the probe according to the embodiment of the invention.

FIG. 27A is a schematic partial side view of a probe and the body of an optical biometer according to an embodiment of the invention.

FIG. 27B is a schematic partial side view of the probe and the body according to the embodiment of the invention.

FIG. 27C is a schematic partial side view of the probe and the body according to the embodiment of the invention.

FIG. 28A is a schematic side view of the end face of a probe according to an embodiment of the invention.

FIG. 28B is a schematic view of the end face of the probe according to the embodiment of the invention.

FIG. 29A is a schematic vertical cross-sectional view of a probe according to another embodiment of the invention.

FIG. 29B is a schematic vertical cross-sectional view of the probe according to the embodiment of the invention.

FIG. 30 is a schematic side view of a probe according to another embodiment of the invention.

FIG. 31 is a schematic view of the end face of a probe according to an embodiment of the invention.

FIG. 32A is a schematic vertical cross-sectional view of a probe and illustrates the problem to be solved by the present invention.

FIG. 32B is a schematic vertical cross-sectional view of the probe and illustrates the problem to be solved by the invention.

FIG. 32C is a schematic vertical cross-sectional view of the probe and illustrates the problem to be solved by the invention.

### DETAILED DESCRIPTION OF EMBODIMENTS

The embodiments of a light emission/reception system and an optical biometer according to the present invention will now be described in reference to the accompanying drawings. However, the scope of the invention is not limited to the disclosed embodiments. The same components are denoted by the same reference numerals without redundant description throughout the embodiments.

Overview of Optical Biometer

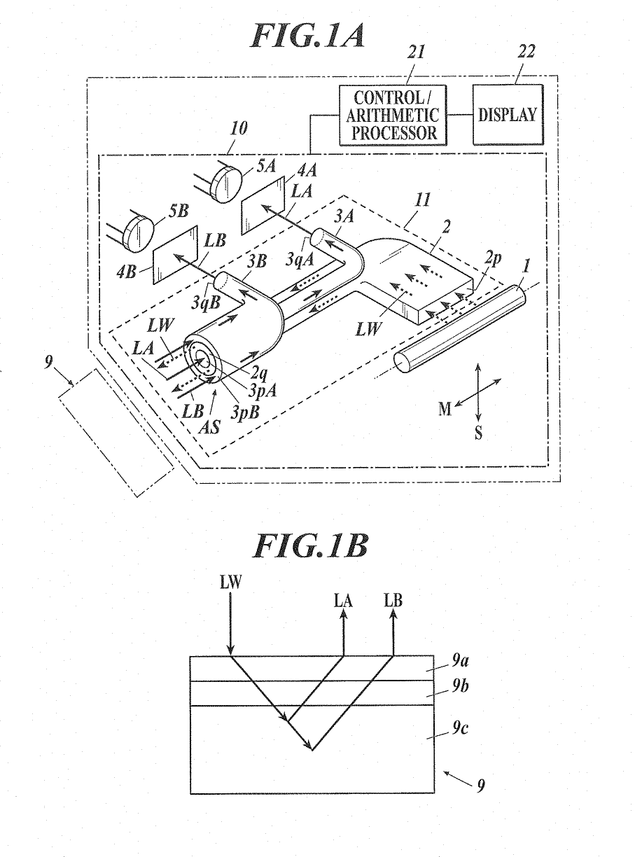

FIG. 1A schematically illustrates configurations of a light emission/reception system and other components according to an embodiment. As illustrated in FIG. 1A, an optical biometer or icteric indicator 20 includes a light emission/reception system 10 and a light guide 11. The light emission/reception system 10 includes a light source or xenon tube 1, two diffuser plates 4A and 4B, and two dual-wavelength photodetectors 5A and 5B. The light guide 11 includes a forward optical fiber bundle 2 that transmits forward light and two backward optical fiber bundles 3A and 3B that transmit reflected light. The forward optical fiber bundle 2 defines a light emitting system that guides white source light beams LW from the xenon tube 1 via a light inlet 2p to a light outlet 2q and radiates the white source light beams LW to a living body 9 (a subject to be examined). The backward optical fiber bundles 3A and 3B define a dual-path light receiving system and respectively guide scattered return light beams LA and LB from the living body 9 via light inlets 3pA and 3pB to light outlets 3qA and 3qB to transmit the beams toward light detection sides or diffuser plates 4A and 4B.

The forward optical fiber bundle 2 and the backward optical fiber bundles 3A and 3B are light guides composed of optical fibers, each having end faces which the light enters or exits. The forward optical fiber bundle 2 and the backward optical fiber bundles 3A and 3B are optically isolated from each other such that the source light from the forward optical fiber bundle 2 inevitably goes through the living body 9 and return to the backward optical fiber bundles 3A and 3B. One of the combination of backward optical fiber bundle 3A, the diffuser plate 4A, and the photodetector 5A and the combination of the backward optical fiber bundle 3B, the diffuser plate 4B, and the photodetector 5B may be omitted to build up a single-path light receiving system.

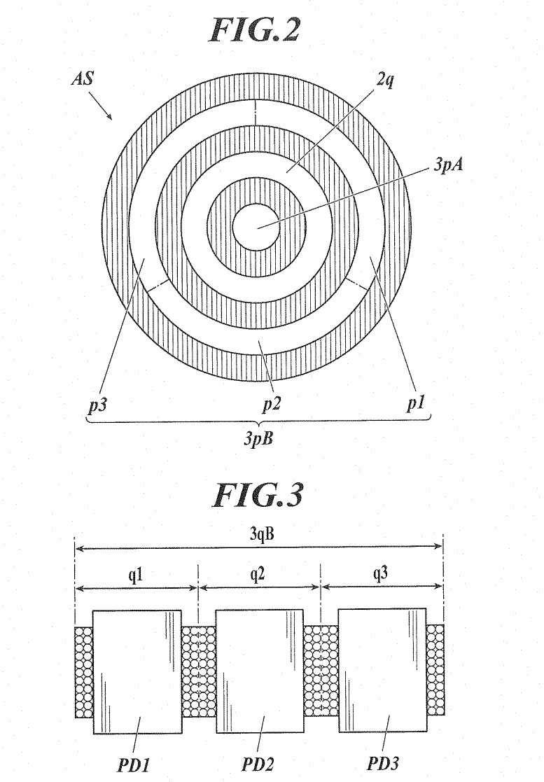

The xenon tube 1 is a long straight light source. Thus, the light inlet 2p of the forward optical fiber bundle 2 has a horizontally rectangular profile to receive source light from the xenon tube 1. FIG. 2 illustrates a schematic view of the end face to come into contact with the surface of the subject at the measurement. As illustrated in FIG. 2, the light outlet 2q of the forward optical fiber bundle 2 is annular. In the cross-sectional view, the light outlet 2q surrounds a circular light inlet 3pA of the backward optical fiber bundle 3A while the light outlet 2q is surrounded by the annular light inlet 3pB of the backward optical fiber bundle 3B. In other words, the light outlet 2q of the forward optical fiber bundle 2 is concentric with the light inlets 3pA and 3pB of the backward optical fiber bundles 3A and 3B. The xenon tube 1 may be replaced with multiple light sources, for example, light emitting diodes (LEDs) disposed in series. The light guide 11 can be used in this case.

The light outlet 2q of the forward optical fiber bundle 2 and the light inlets 3pA and 3pB of the backward optical fiber bundles 3A and 3B reside at the end face AS of the probe of the icteric indicator 20. The end face AS of the probe is urged onto the living body 9, for example, onto the forehead or breast of a newborn infant, and the xenon tube 1 radiates white source light for measurement. The white source light beams LW from the xenon tube 1 enter the light inlet 2p and travel through the forward optical fiber bundle 2. The white source light beams are then guided to the light outlet 2q and radiated in a circular flux toward the living body 9.

As illustrated in FIG. 1B, the white source light beams LW, which enter the living body 9 through epidermis 9a, pass through dermis 9b and then scattered in a subcutaneous tissue 9c. The backscattered white source light beams LW exit the living body 9 and enter the light inlets 3pA and 3pB as scattered return light beams LA and LB. As illustrated in FIG. 1A, the scattered return light beams LA and LB from the living body 9 are respectively guided from the light inlets 3pA and 3pB of the backward optical fiber bundles 3A and 3B to the light outlets 3qA and 3qB and exit the light outlets 3qA and 3qB. The scattered return light beams LA and LB are diffused through the diffuser plates 4A and 4B, respectively to remove the uneven brightness. The scattered return light beams LA and LB are then received by the dual-wavelength photodetectors 5A and 5B, respectively.

The dual-wavelength photodetectors 5A and 5B each have green and blue filters on the light reception face to detect the intensities of the blue band having a central wavelength at 450 nm and the green band having a central wavelength at 550 nm in the transmitted light beams. The intensity of the yellowish color is measured from the intensity of the blue light relative to the intensity of the green light or the ratio of the intensity of the blue light to that of the green light. For example, a low intensity of the blue light relative to the intensity of the green light signifies a high intensity of yellowish color, and determines that the severity of the jaundice is high. In other words, the intensity of the yellowish color of the bilirubin present in the subcutaneous tissue 9c of the living body 9 corresponds to a difference in the optical density between the wavelength regions of the two spectral colors of blue and green. A light receiving system of a dual-path type as in the present embodiment can calculate the difference in the observed results obtained from the two light paths regardless of the color and thickness of the epidermis 9a. The severity of the jaundice can accordingly be more precisely measured.

Embodiments of Light Emission/Reception System

Based on the overview of the biometer described above, embodiments of a light emission/reception system will now be described that includes, for example, a light receiver having multiple segments.

### First Embodiment

As illustrated in FIG. 2, the scattered-light inlet 3pB of the backward optical fiber bundle 3B consists of three light receiver segments p1, p2, and p3, among the light inlets 3pA and 3pB of the backward optical fiber bundles 3A and 3B. The light outlet 2q of the forward optical fiber bundle 2 serves as a light radiator at the end face AS.

The light receiver segments p1, p2, and p3 are in different angular ranges around the center axis of the light outlet 2q which is the light exit. As illustrated in FIG. 2, the light receiver segments p1, p2, and p3 are disposed at substantially equal intervals with a central angle of 120°. Thus, the light receiver segments p1, p2, and p3 are in different angular ranges around the central axis.

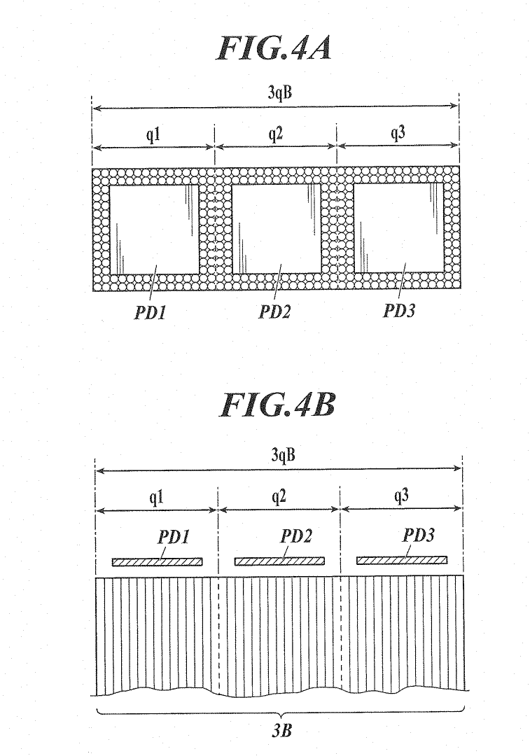

As illustrated in FIG. 3, FIG. 4A, and FIG. 4B, the light outlet 3qB of the backward optical fiber bundle 3B consists of three divided segments, i.e., a light outlet segment q1 in communication with a light receiver segment p1, a light outlet segment q2 in communication with a light receiver segment p2, and a light outlet segment q3 in communication with a light receiver segment p3. In the drawings, the light outlet segments q1, q2, and q3 are disposed in parallel.

Instead of the dual-wavelength photodetectors 5A and 5B, photodetectors PD 1, PD 2, PD 3 are respectively disposed in the light outlet segments q1, q2, and q3 as illustrated in FIG. 3 or FIG. 4A and FIG. 4B. The scattered return light from the light outlet segment q1 enters the photodetector PD 1; the scattered return light from the light outlet segment q2 the photodetector PD 2; and the scattered return light from the light outlet segment q3 the photodetector PD 3. The photodetectors PD 1, PD 2, and PD 3 independently detect the light. In other words, the photodetectors PD 1, PD 2, and PD 3 are disposed in different segments for independent detection of the light. In this embodiment, three segments are disposed. Thus, the three light receiver segments p1, p2, and p3 for independent measurement of the light are disposed at the end face AS. The light receiver segments p1, p2, and p3 are disposed in different angular ranges around the center axis of the light radiator. Depending on the tilt of the probe P relative to the living surface or the uneven contact of the end face AS with the living body, the light intensity detected in the segments are different. The level of uniformity of the contact of the end face AS with the surface of the living body can be detected based on the intensity detected in the segments. This configuration can improve the precision or reproducibility and the accuracy of the measurement. The details of the measurement will be described below.

The light detecting component according to the present embodiment consists of photosensors (for example, photodiodes) corresponding to the segments, such as the photodetectors PD 1, PD 2, and PD 3.

### Second Embodiment

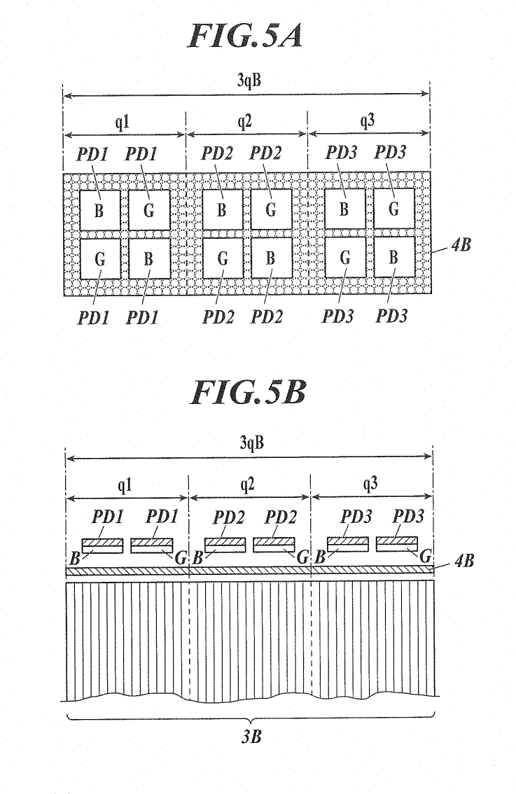

As illustrated in FIG. 5A and FIG. 5B, a second embodiment may include green filters G and blue filters B that are not shown in FIG. 3, FIG. 4A, and FIG. 4B. The diffuser plate 4B may be disposed in the same manner as FIG. 5A and FIG. 5B.

In the embodiment illustrated in FIG. 5A and FIG. 5B, two green filters G and two blue filters B are alternated. This arrangement can reduce the unevenness in intensity between the blue light and green light received at different positions. The numbers of alternated blue filters and green filters may be each three or more for each segment.

Four filters (two green filters and two blue filters) are disposed for each segment. In other words, the photodetectors PD 1, PD 2, or PD 3 each are provided with four filters. The photodetectors PD 1, PD 2, and PD 3 are thus provided with twelve photodetectors in total.

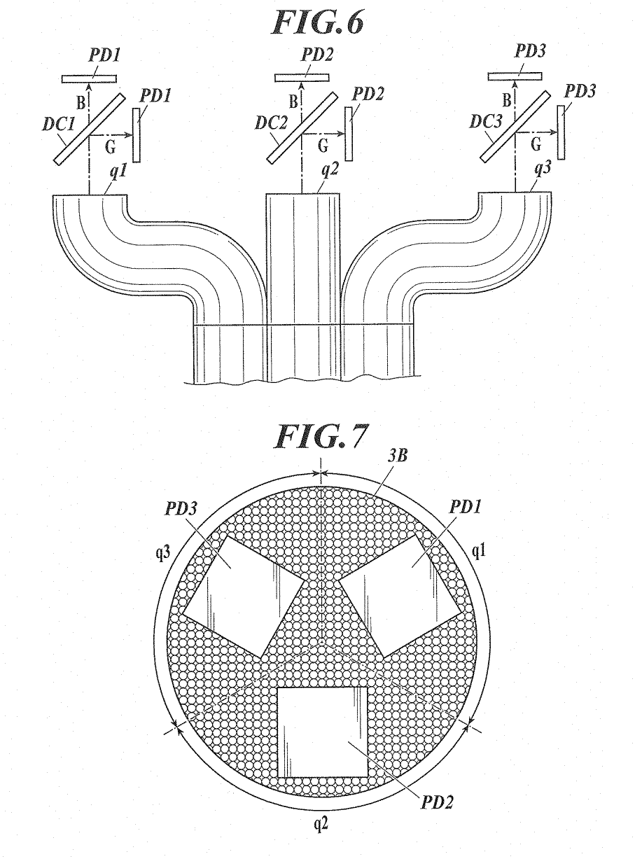

In case that no green filter G and no blue filter B are disposed in the light outlets as illustrated in FIG. 3, FIG. 4A, and FIG. 4B, light may be measured in a time-divisional scheme; green light is radiated for measurement of the intensity of the green light, and blue light is radiated for measurement of the intensity of the blue light. As illustrated in FIG. 6, spectroscopes, for example, dichroic mirrors DC1, DC2, and DC3 may be disposed in front of the light outlet segments q1, q2, and q3 and the photodetectors PD 1 (PD 2 and PD 3) may be disposed in the optical paths of the spectral light.

The light outlet segments q1, q2, and q3 in FIG. 3, FIG. 4A, FIG. 4B, FIG. 5A, and FIG. 5B are rectangular. Alternatively, the light outlet segments q1, q2, and q3 may have any other shape.

### Third Embodiment

As illustrated in FIG. 7, the light outlet segments q1, q2, and q3 according to a third embodiment are circumferentially disposed in the respective segments in the backward optical fiber bundle 3B. In this embodiment, three fan-shaped light outlets each having a central angle of 120° are disposed. The photodetectors PD 1, PD 2, and PD 3 are disposed in the light outlet segments q1, q2, and q3 as in the first and second embodiments. For example, filters, time-divisional light radiation schemes, and dichroic mirrors are used as required.

### Fourth Embodiment

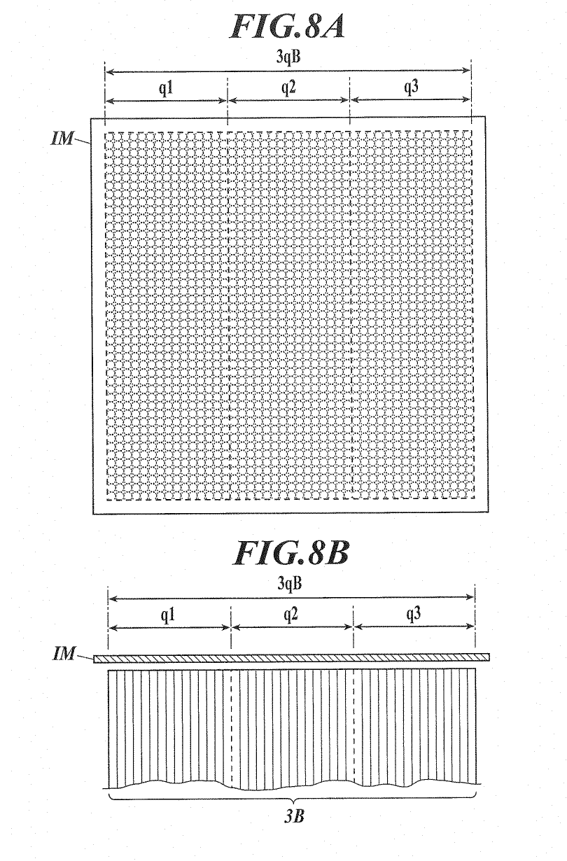

As illustrated in FIG. 8A and FIG. 8B, the light detector according to a fourth embodiment is a two-dimensional image sensor IM extending over the light outlet segments q1, q2, and q3. The ranges of pixels for reception and measurement of return light from the light outlet segments q1, q2, and q3 are preferably predetermined. Filters and time-divisional light radiation schemes are used as required.

### Fifth Embodiment

The backward optical fiber bundle 3B may diverge into the light outlet segments q1, q2, and q3 as illustrated in FIG. 6 or may be physically divided with partitions. This can reduce the mixing of scattered return light at the photodetectors PD 1, PD 2, and PD 3. It should be noted that some mixing of light beams (less efficient optical separation in the segments) is allowable in principle; the directional conditions of the light outlet segments depend on the angles of the segments around the light radiator and are affected by the tilt of the probe P relative to the surface of the living body or the uneven contact of the end face AS with the surface of the living body, resulting in difference among light intensities detected by the photodetectors PD 1, PD 2, and PD 3 in front of the respective segments.

### Sixth Embodiment

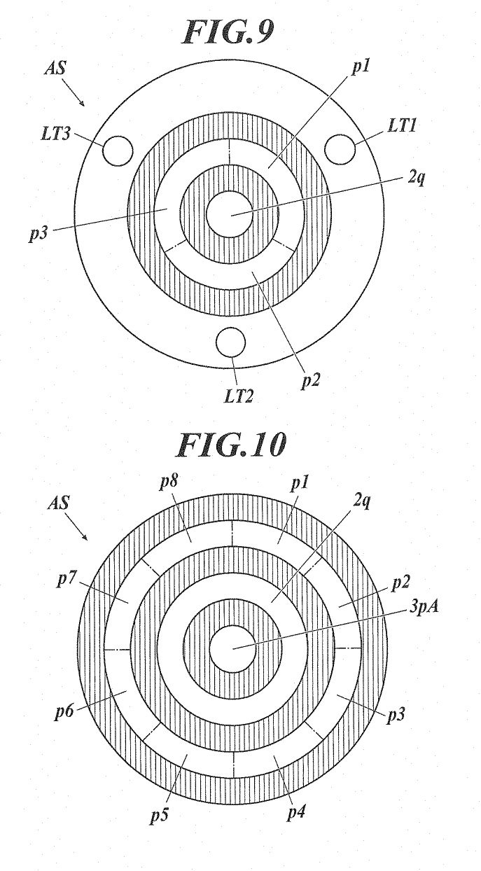

As illustrated in FIG. 9, the light receiver segments p1, p2, and p3 according to a sixth embodiment surround the light outlet 2q at the end face AS. The light receiver segments p1, p2, and p3 are provided with illumination light emitters LT1, LT2, and LT3 in the neighborhood. The illumination light emitters LT1, LT2, and LT3 at the end face AS may be LEDs.

The illumination light emitters LT1, LT2, and LT3 can serve as light sources for preliminary light emission. For this purpose, the wavelengths of the light beams from the illumination light emitters LT1, LT2, and LT3 are equal to the wavelengths of the scattered return light beams for ready assessment of the effects of the wavelengths on the measurement.

The illumination light emitters LT1, LT2, and LT3 can serve as lamps for work in the darkness, in particular, for visual inspection of the target surface of the living body onto which the end face AS is urged.

### Other Embodiments

In FIG. 2 and FIG. 9, the light receiver segments p1, p2, and p3 are divided with partitions into three segments and each have a central angle of 120°. Alternatively, the light receiver segments p1, p2, and p3 may have different central angles.

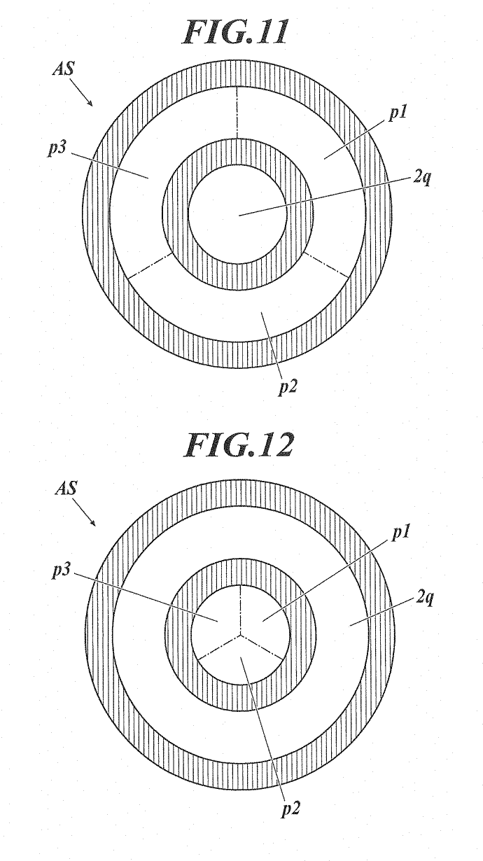

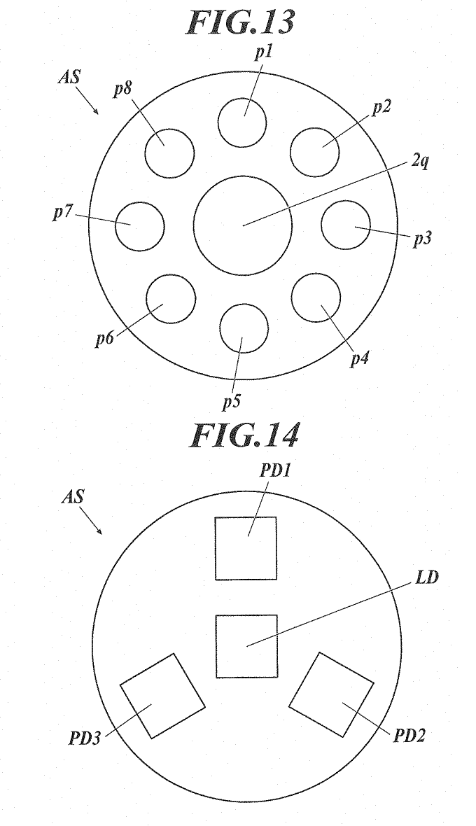

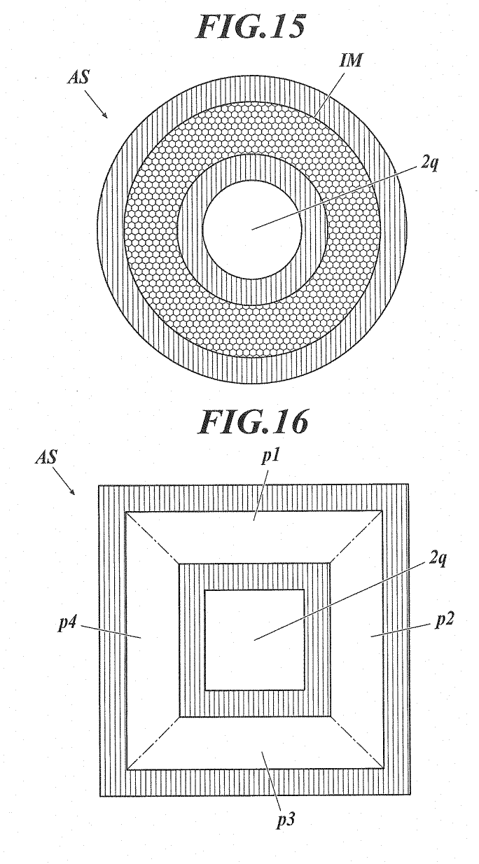

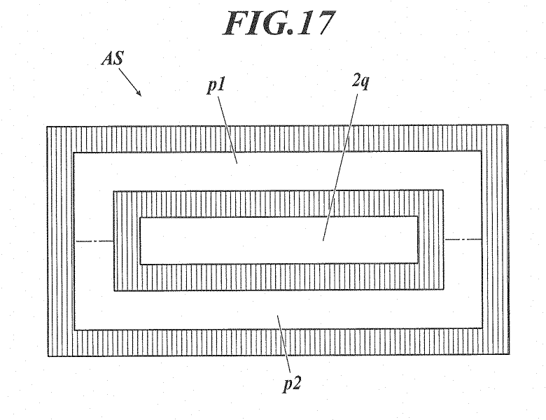

The light reception system may include any of one optical path (in FIG. 9, FIG. 11, FIG. 12, FIG. 13, FIG. 14, FIG. 15, FIG. 16, and FIG. 17), two optical paths (in FIG. 2 and FIG. 10), and three or more optical paths (not shown). In the two optical paths, the light inlet 3pB remote from the center of the end face AS may have multiple segments or the light inlet 3pA adjacent to the center of the end face AS may have multiple segments. Alternatively, both the outer and inner inlets 3pB and 3pA may have multiple segments.

The light reception system may include four or more segments (in FIG. 10) instead of the three segments (in FIG. 2). For example, eight light receiver segments p1 to p8 are disposed in FIG. 10. An odd number or even number of segments may be disposed. The light receiver segments p1, p2, and p3 may be annular (in FIG. 2, FIG. 9, FIG. 10, FIG. 11, and FIG. 15), circular (in FIG. 12), hollow square or rectangular (in FIG. 16 and FIG. 17), or solid square or rectangular (not shown).

As illustrated in FIG. 13, the light receiver segments p1 to p8 may be circumferentially disposed around the light outlet 2q at intervals. In FIG. 13, the light receiver segments p1 to p8 are each circular (any other shape is possible) and are circumferentially disposed around the light outlet 2q at intervals. As illustrated in FIG. 14, light receiver segments PD1 to PD3 may be circumferentially disposed around a light radiator LD at intervals. In FIG. 14, the light radiator LD at the end face AS is a light emitting element. In FIG. 14, the light receiver segments PD 1 to PD 3 at the end face AS are photodetectors for the respective segments. The light radiator LD at the end face AS may be combined with the backward optical fiber bundles 3A and 3B, or the photodetectors PD 1 to PD 3 at the end face AS may be combined with the forward optical fiber buddle 2.

One or more photodetectors may be disposed for each segment in the light reception system.

As illustrated in FIG. 15, the light receiver segment may be provided with a two-dimensional image sensor IM at the end face AS. In FIG. 15, the two-dimensional image sensor IM is annular and surrounds the light radiator or the light outlet 2q. Without limitation to this configuration, the light radiator or the light outlet 2q may be annular to surround a circular two-dimensional image sensor array.

In FIG. 16, the light radiator or outlet 2q is hollow square and the light receiver segments p1-p4 form a hollow rectangular shape. In FIG. 17, the light radiator or the light outlet 2q and the light inlet 3pB are hollow rectangular. In this manner, any shape may be selected, and any polygon, for example, hexagon and octagon may be selected. The number of corners may be the same as or different from the number of segments.

The forward optical fiber bundle 2 and the backward optical fiber bundles 3A and 3B may be replaced with light guides, light pipes, or combination thereof having the same functions.

### Embodiments of Measurement Schemes

As illustrated in FIG. 1A, the optical biometer or the icteric indicator 20 includes a control/arithmetic processor 21 that controls the radiation of source light for jaundice measurement from the xenon tube 1 to the light radiator or outlet 2q and calculates the observed results from the intensity of the return light detected at the light receiver segments (3pA and 3pB); and a display 22 that presents, for example, the calculated value.

The control/arithmetic processor 21 performs the control and calculation according to any of the following schemes.

First Measurement Scheme

In accordance with a first measurement scheme, the control/arithmetic processor 21 calculates an observed result from the light intensities with proviso that the difference among the light intensities detected in the segments falls within a predetermined threshold.

The control/arithmetic processor 21 instructs the light radiator to radiate the source light toward the living body and the light receiver segments p1, p2, and p3 to receive the scattered return light from the living body. If the difference among the light intensities falls within a predetermined threshold, the observed results are calculated from the light intensity and presented on the display 22. Since multiple light intensities are detected, operations, for example, averaging procedures, may be employed. The light intensity detected in any one of the segments may be selected for calculation of an observed result. The difference falling within the predetermined threshold indicates that the tilt of the probe P relative to the surface or the contact of the end face AS with the surface is kept at a predetermined level, resulting in calculation of a correct value.

In this scheme, the control/arithmetic processor 21 instructs the light radiator to reradiate the source light if the difference among the light intensities does not fall within the predetermined threshold. The light radiator repeats the radiation of the source light until the difference falls within the predetermined threshold.

The light intensities from which the difference is calculated may be ones detected by the light receiver segments p1, p2, and p3 or parameters reflecting the intensity. This holds in other measurement schemes.

Second Measurement Scheme

In accordance with a second measurement scheme, the control/arithmetic processor 21 controls preliminary radiation of source light. When the difference among the light intensities detected in the segments after the preliminary radiation falls within the predetermined threshold, the control/arithmetic processor 21 controls true radiation of the source light and calculates the observed results from the light intensities detected at the light receiver segments p1, p2, and p3.

The illumination light emitters LT1, LT2, and LT3 or the xenon tube 1, a light source for measurement, can serve as a light source for the preliminary emission of the source light. The preliminary radiation of the source light from the xenon tube 1 is performed under a radiating condition different from that for the true radiation, for example, the radiation of the source light with reduced intensity. The preliminary emission of the source light from the illumination light emitters LT1, LT2, and LT3 (LEDs) or the preliminary radiation of the source light with reduced intensity by the xenon tube 1 can reduce the power consumption before the true radiation.

The illumination light emitters LT1, LT2, and LT3 serving as light sources for preliminary emission of the source light may be operated in response to the start switch or a dedicated switch driving the illumination light emitters LT1, LT2, and LT3. The dedicated switch allows the illumination light emitters LT1, LT2, and LT3 to emit light at different cycles or for different periods and serve as luminaires for work in the darkness.

### Third Measurement Scheme

In accordance with a third measurement scheme, the control/arithmetic processor 21 uses incident external or environmental light for a light source instead of the preliminary light radiation to obtain a dark count value at a non-radiation time. The control/arithmetic processor 21 instructs the xenon tube 1 to control true radiation of the source light in response to the difference in intensity of the return light detected in the segments falling within a predetermined threshold. The control/arithmetic processor 21 then calculates the observed results from the intensity of the return light detected by the light receiver segments p1, p2, and p3 after the true radiation.

The unevenness over the positions of the incident external light can determine the tilt of the probe P or the uneven contact of the end face AS with the surface BS.

In the case that no external light is available or the end face AS is in close contact with, for example, a soft skin despite the tilting probe P, the external beams cannot be detected. In such a case, the second measurement scheme is employed to instruct the illumination light emitters LT1, LT2, and LT3 or the light source (xenon tube 1) to emit light. These light emitters mounted to the icteric indicator can precisely determine the tilt of the probe P or the uneven contact of the end face AS.

First Supplementary Function

The control/arithmetic processor 21 may have a supplementary function that instructs the display 22 to present that the difference in intensity of the return light detected in the segments exceeds a predetermined threshold.

The display may be a flat panel display or a dedicated LED display. The flat panel display capable of presenting characters and illustrations may present textual data, such as “The measurement probe is tilting relative to the living body” and “Keep the measurement probe vertical to the measured face”. Alternatively, the flat panel display may present graphic data indicating such messages.

The display 22 may include an audio output device (speaker) to inform a user of the tilt of the probe P through a voice or beep.

This alerting or informing function may be executed simultaneously with tentative display of the observed results even in the case that the difference exceeds the threshold. The observed results in the case that the difference exceeds the threshold can be displayed with an alerting indicator. The observed results in the case that the difference falls within the threshold can be displayed with an indicator of the normal state. Either or both of such displaying modes can be selected.

In the case that the difference exceeds the threshold, only an alert may be issued without display of the observed results.

### Second Supplementary Function

The control/arithmetic processor 21 may have a supplementary function that instructs the display 22 to present the tilting direction of the probe P relative to the surface of the living body or the direction for correction of the tilt.

To execute this supplementary function, the icteric indicator 20 in FIG. 18A and FIG. 18B includes a display 22B, such as a flat panel display or LED display, on the top panel remote from the end face AS. The display 22B may present a centering instruction 23 according to the first supplementary function and directional instructions 24a to 24d according to the second supplementary function. The icteric indicator 20 also includes a main display 22A or flat panel display on the front side in addition to the display 22B on the top panel. The main display 22A presents, for example, the observed results. The displays 22A and 22B may be integrated. In the case that the flat panel display 22B on the top panel can present the observed results with numerals and characters, the display 22A on the front side can be omitted. In the case that the display 22A can present the centering instruction 23 and the directional instructions 24a to 24d, the display 22B on the top panel can be omitted as illustrated in FIG. 18C. Even in such a case, both the display 22A on the front side and the display 22B on the top panel may be provided to present the centering instruction 23 and the directional instructions 24a to 24d.

The audio outputting device or speaker of the display 22 may inform the user of the tilting direction of the probe P relative to the surface or the direction for correction of the tilting.

The first and second supplementary functions can be combined with the first and second measurement schemes to guide the user to quickly obtain observed results. The first and second supplementary functions can also be combined with the following measurement schemes to improve the precision of the measurement.

Fourth Measurement Scheme

If the difference in intensity of the return light in the segments exceeds a predetermined threshold, the control/arithmetic processor 21 may calculate an observed result from the intensity of return light detected in only some of the segments according to a fourth measurement scheme.

For example, the control/arithmetic processor 21 calculates the observed result only from the intensity detected in specific light receiver segments where received return light has a high intensity. The receiver segments where the received return light has a high intensity are in close contact with the surface and thus receive more return light scattered from the surface. Despite the probe P tilting relative to the surface or the end face AS being in imperfect contact with the end face AS, the control/arithmetic processor 21 calculates the observed results from the intensity of the return light detected in the light receiver segments at the end face AS that are in close contact with the surface among the light receiver segments p1, p2, and p3. Precision of the measurement is thereby improved.

Alternatively, the control/arithmetic processor 21 calculates an observed result only from the intensity of return light at a relatively similar level in the specific segments. The light intensity at a relatively similar level indicates that the segments on the end face AS are in substantially even contact with the surface. The light intensity shifted from the others or the light intensity detected in the segment at the end face AS in uneven contact with the surface is excluded. Then, the observed result is calculated only from the light intensity detected in the segments at the end face AS in even contact with the surface. The precision of the observed result is thereby improved.

In this scheme, the control/arithmetic processor 21 may instruct the display 22 to represent the low level of reliability of the observed result due to the difference in the return light exceeding the predetermined threshold between the segments. For example, the difference exceeding a first threshold is represented by “Measurement Reliability Level: Medium” and the difference exceeding a second threshold larger than the first threshold is represented by “Measurement Reliability Level: Low”.

The user may redetermine the intensity of the return light as required. The first and second supplementary functions can correct the orientation of the icteric indicator 20 to obtain an observed result corresponding to “Measurement Reliability Level: High”.

Fifth Measurement Scheme

In accordance with a fifth measurement scheme, the control/arithmetic processor 21 instructs predetermined cycles of sequential radiation of the source light. The control/arithmetic processor 21 calculates a difference in intensity of return light detected in the segments for each radiation. The control/arithmetic processor 21 calculates an observed result from the relatively even intensity of the return light detected in the light receiver segments.

Unlike the first and second measurement schemes, the fifth measurement scheme does not involve calculation of the difference for each radiation. A set of sequential light beams is radiated at a predetermined time rate, followed by the calculation of the difference for each radiation. The difference calculated from the relatively even light intensities detected in the light receiver segments can achieve a high precision of the measurement.

In this scheme, the user corrects the orientation of the icteric indicator 20 with reference to the indication on the display 22 as described in the first and second supplementary functions to achieve a higher precision of measurement.

This scheme can use a light source suitable for sequential emission of light beams, for example, an LED or a laser emitter.

### Sixth Measurement Scheme

In accordance with a sixth measurement scheme, the control/arithmetic processor 21 calculates a difference among light intensities detected in a plurality of segments and performs the correction of the detected value or corrects the effect of the tilt of the probe and the uneven contact of the end face with the surface to calculate a more accurate observed result. The effect of the tilting probe and the contact of the end face with the surface is preliminarily examined, in other words, the correlation of an offset with the difference above is determined. Arithmetic expressions including the correlation can be implemented in the icteric indicator 20 for offsetting of the difference.

Miscellaneous

Further description will be provided below.

Variation (a): In FIG. 2, the optical fibers in the light receiver segments p1, p2, and p3 may have any angular ranges that corresponds to the angular ranges of the light outlet segments q1, q2, and q3.

The angular ranges in the light inlet at the end face AS of the backward optical fiber bundle 3B may be maintained in the outlet or converted into distribution on a specific coordinate. Alternatively, the angular ranges in the light inlet may be converted at random in each of the segments.

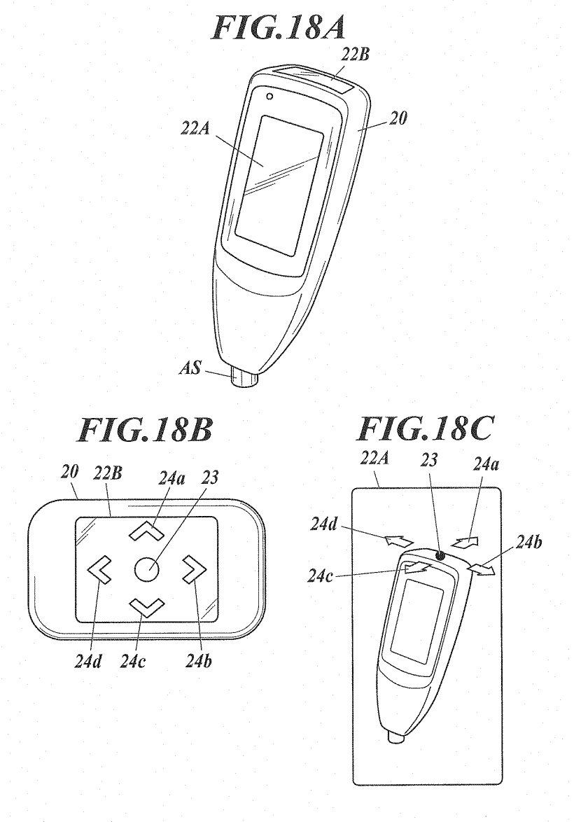

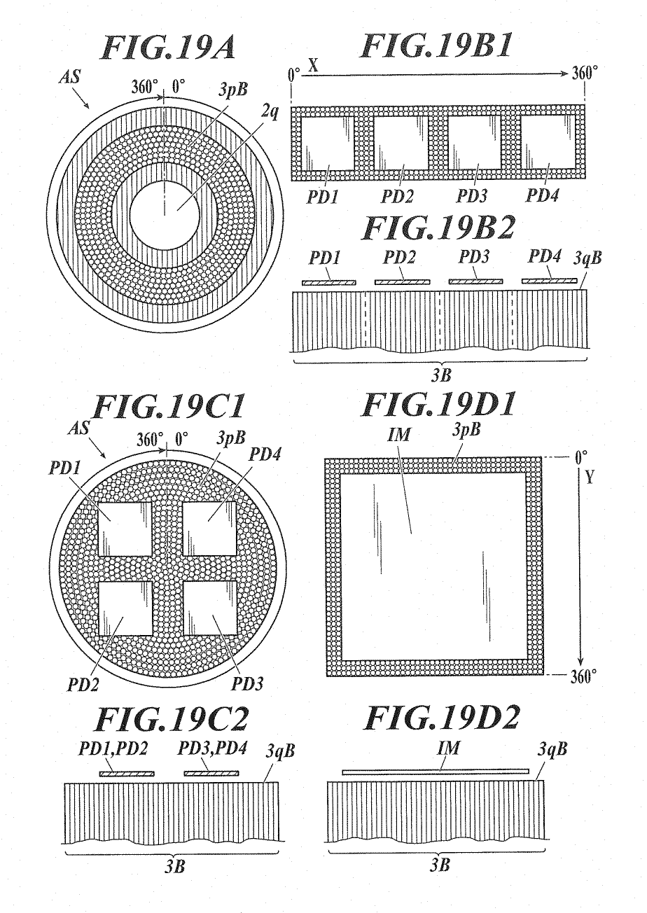

As illustrated in FIG. 19A, the light inlet 3pB is circumferentially disposed around the light outlet 2q at the end face AS. As illustrated in FIG. 19B1 and FIG. 19B2, the angular ranges in the light outlet 3qB of the backward optical fiber bundle 3B is converted into distribution on the X coordinate at the light outlet. The photodetectors PD 1, . . . , PD 4 are also disposed along the X coordinate and can detect scattered return light beams received in the respective angular areas on the end face AS.

In FIG. 19C1 and FIG. 19C2, the light inlet 3pB has a cross-section similar to that in FIG. 19A and is circumferentially disposed around the light outlet 2q at the end face AS. The light outlet 3qB in the backward optical fiber bundle 3B has the same cross-section around the central axis. The photodetectors PD 1, . . . , PD 4 are disposed in different angular ranges and can detect the scattered return light beams received in the angular areas on the end face AS. In this case, the backward optical fiber bundle 3B may be an image fiber having the same optical fiber arrangement both at the light inlet and the light outlet.

In FIG. 19D1 and FIG. 19D2, the light inlet 3pB has different angular ranges from that around the central axis of the light outlet 2q at the end face AS in FIG. 19A. The light outlet 3qB of the backward optical fiber bundle 3B has angular ranges converted into distribution on the Y coordinate at the exit end. The photodetectors are also disposed along the Y coordinate corresponding to, for example, pixels of the two-dimensional image sensor IM and can detect scattered return light beams in different angular ranges on the end face AS.

Separately from the design of the optical fiber bundle, the number of photodetectors can be determined and the number of segments at central angles can thereby be appropriately adjusted. In particular, a two-dimensional image sensor can acquire the continuously differing light intensities as continuous distributions, unlike other sensors acquiring the light intensities as integrated values of light beams detected in the respective areas. The uneven contact of the end face AS with the surface of the living body depending on the angles of the probe P can be more locally detected.

Variation (b): The above-mentioned configurations each include a light radiation/reception system having multiple detection channels disposed at angles around the axis of the light radiator. The segments cover different angular areas for detection of light intensity on the end face AS of the probe P. The target areas for detection on the end face AS are different among the detection channels.

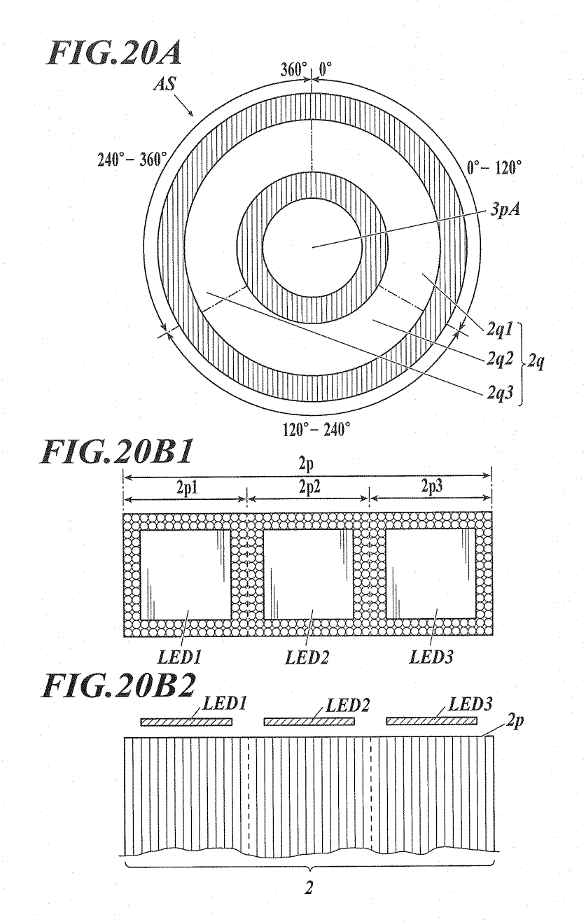

The light outlet may be divided into multiple segments around the circumference on the end face AS of the probe P, instead of the light inlet. In FIG. 20A, the light outlet 2q is divided in three segments 2q1, 2q2, and 2q3 from which light beams are radiated. The segments 2q1, 2q2, and 2q3 of the light outlet 2q are disposed at different angular areas around the light inlet 3pA. As illustrated in FIG. 20B1 and FIG. 20B2, light emitting elements LED 1, LED 2, and LED 3 are disposed in the light inlet 2P divided in segments 2p1, 2p2, and 2p3 corresponding to the light radiator segments 2q1, 2q2, and 2q3.

In the case that light inlet 3pA has one segment and the light outlet has multiple segments, the control/arithmetic processor 21 controls the time-divisional radiation of light beams through the light radiator segments 2q1, 2q2, and 2q3. The scattered return light beams enter the light inlet 3pA, and then the light intensity from different segments are detected. This configuration also allows the independent detection of light intensity in the separate channels at different angular areas on the end face AS of the probe P.

This measurement scheme may involve preliminary time-divisional radiation of light beams from the light radiator segments 2q1, 2q2, and 2q3 and simultaneous true radiation of light beams from the light radiators 2q1, 2q2, and 2q3, resulting in detection of the scattered return light. Alternatively, an observed result may be computed from the intensity of time-divisionally radiated source light.

Instead of the light inlet divided in two or more segments, the light outlet may be divided in multiple segments. In both the cases, light emitting elements may be provided in the light inlet segments.

Multiple light receiver segments may require the same number of photodetectors, resulting in increases in manufacturing costs and/or dimensions of the product. In order to provide a sufficient light intensity, multiple light sources, for example, LED light sources disposed adjacent to the light radiator segments may be utilized for other purposes.

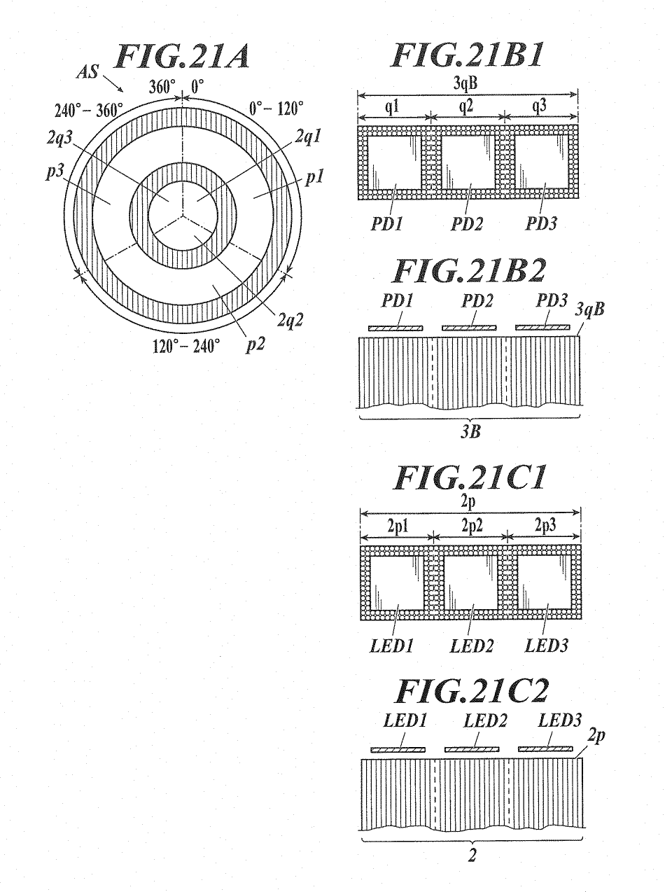

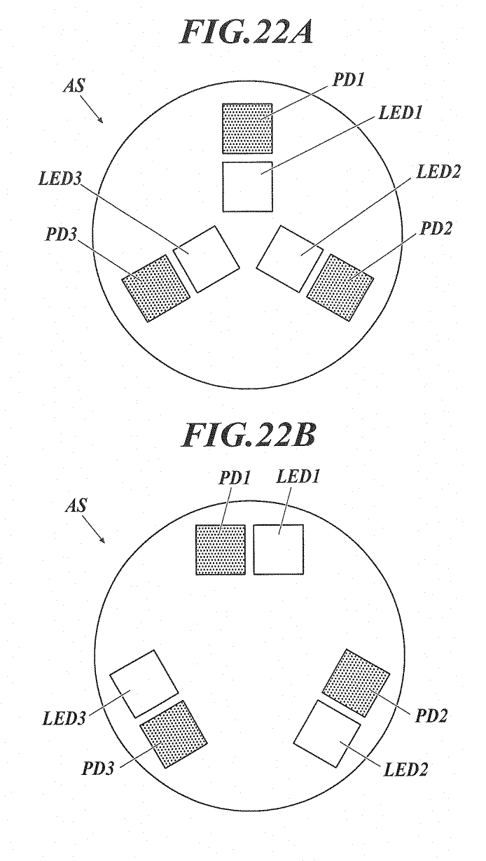

Variation (c): As illustrated in FIG. 21A, FIG. 22A, and FIG. 22B, both of multiple light receiver segments and light radiator segments may be disposed at different angular areas.

This measurement scheme may involve simultaneous radiation of source light from the light radiator segments and subsequent detection of the scattered return light in the light receiver segments. Alternatively, the measurement scheme may involve the time-divisional radiation of light as described in Variation (b). Preliminary light may be time-divisionally radiated and the true light may be simultaneously radiated.

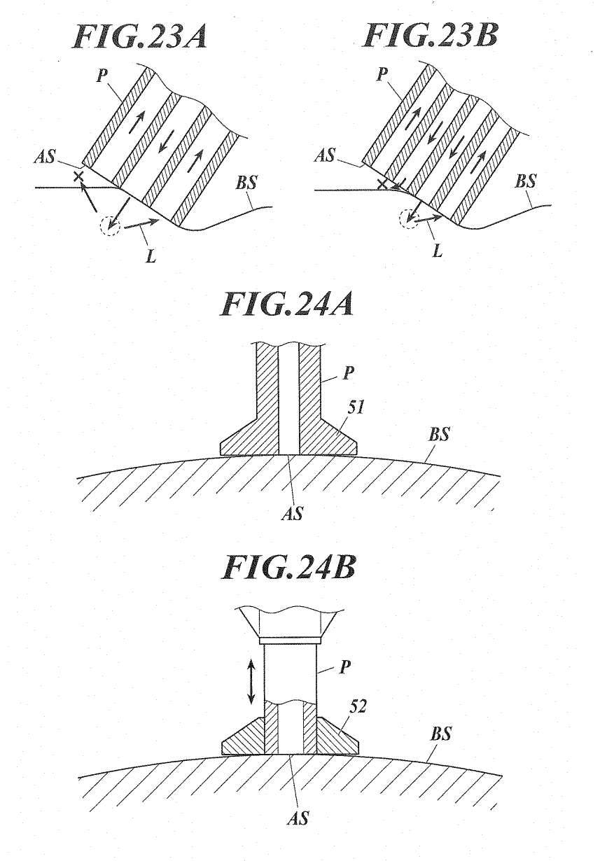

Compared to the embodiment in which only the light outlet is divided in multiple segments in FIG. 23A, the embodiment in which both the light outlet and the light inlet are divided in multiple segments in FIG. 23B allows radial separation of angular areas for detection of the return light on the end face AS but is readily affected by the uneven contact of the end face AS with the surface BS due to the tilt of the probe P. For example, the tilt of the probe P may hamper the close contact of the light radiator segments with the living body. The source light cannot readily enter the living body on the partial end face AS that is not in contact with the surface BS even at a slight tilt of the probe P in some cases.

Other Solutions

Other solutions will now be described for a reduction in or the detection of the tilt of the probe P or the unevenness of the contact of the end face AS with the surface BS. The solutions that will be described below do not use an optical measurement system.

Solution (1): The solution for a reduction in the tilt of the probe P and the unevenness of the contact of the end face AS with the surface BS will now be described.

The probe P in FIG. 24A has a flaring end 51, i.e., an end face AS with a wider area. This prevents the probe P from tilting relative to the surface BS.

As illustrated in FIG. 24B, the probe P may be mounted to a bottom-end piece 52 at the end for the same purpose. The probe P is inserted into a hole in the center of the bottom-end piece 52 and is held at a constant angle.

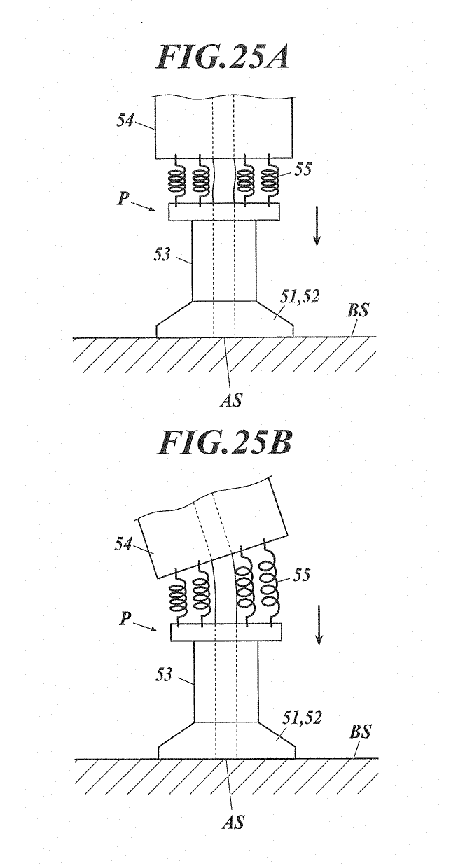

As illustrated in FIG. 25A, an end cap 53 of the probe 53 is coupled to a main body casing 54 remote from the end face AS with, for example, springs 55. Thus, the end cap 53 including the end face AS is resiliently and 360° tiltably supported on the main body casing 54. A user may take the probe P at the main body casing 54 in his/her hand and non-vertically urge the probe P onto the surface BS. As illustrated in FIG. 25B, the main body casing 54 tilts or flexes relative to the end cap 53. Thus, the tilt of the probe P is corrected such that the end face AS can be kept in close contact with the surface BS. The end cap 53 in combination with the flaring end 51 or the bottom-end piece 52 can improve the evenness of the contact of the end face AS with the surface BS.

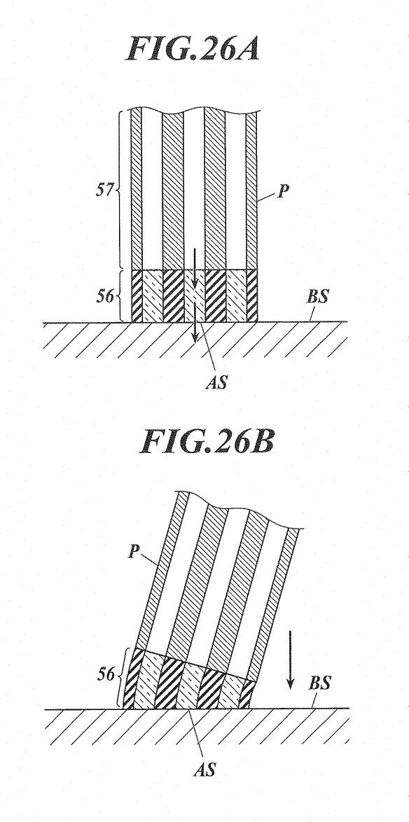

As illustrated in FIG. 26A, the close contact of the end face AS with the surface BS can be achieved by the probe P having an end portion 56 composed of a resilient member, instead of the springs 55. The resilient end portion 56 is more flexibly deformable than a rigid segment 57 remote from the end face AS. As illustrated in FIG. 26B, the rigid segment 57 of the probe P tilting relative to the surface BS does not prevent the resilient end portion 56 from standing on the surface BS. Thus, the end face AS is kept in close contact with the surface BS. The resilient end portion 56 may be composed of, for example, elastomer. In the case of use of optical fibers, the optically insulated member accommodating the optical fibers is composed of a resilient member. A highly flexible optical fiber may be selected.

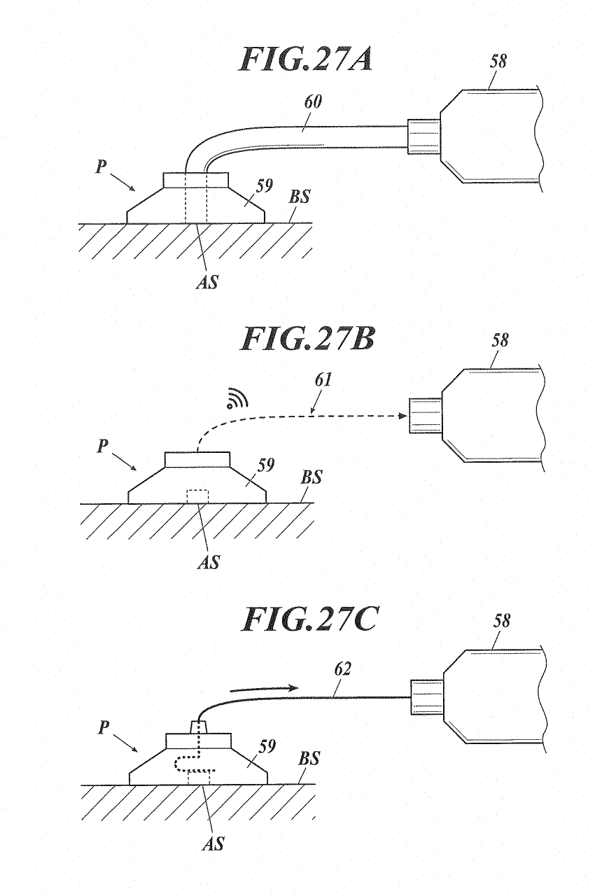

A large, elongated, or heavy icteric indicator is likely to tilt. In such a case, the main body of the icteric indicator and the probe may be separated. A relatively small lightweight optical probe unlikely to tilt relative to a living body can achieve the close contact of the end face AS with the living body, resulting in an enhanced precision of measurement. As illustrated in FIG. 27A, FIG. 27B, and FIG. 27C, a main body 58 can be connected with an end piece 59 of the probe P through, for example, an optical fiber cable 60, a wireless communication link 61, or an electric cable 62.

As in FIG. 24A, FIG. 24B, FIG. 25A, and FIG. 25B, the end piece 59 flares for close contact of the end face AS with the surface BS.

An optical fiber cable 60 consists of, for example, fibers and an outer tube composed of flexible materials.

For use of the wireless communication link 61, an electric circuit is disposed on the end piece 59 to convert the intensity of received scattered return light into an electric signal and wirelessly transmit the signal to the main body 58.

For use of the electric cable 62, an electric circuit is disposed on the end piece 59 to convert the intensity of received scattered return light into an electric signal and transmit the signal to the main body 58 through the electric cable 62.

Solution (2): The solution for detection of tilt of the probe P and the unevenness of the contact of the end face AS with the surface BS will now be described. In response to the detection of the tilting or unevenness, remeasurement, alert or notice or correction of the measurement may be performed. The precision or reproducibility and accuracy of the observed results can be thereby improved.

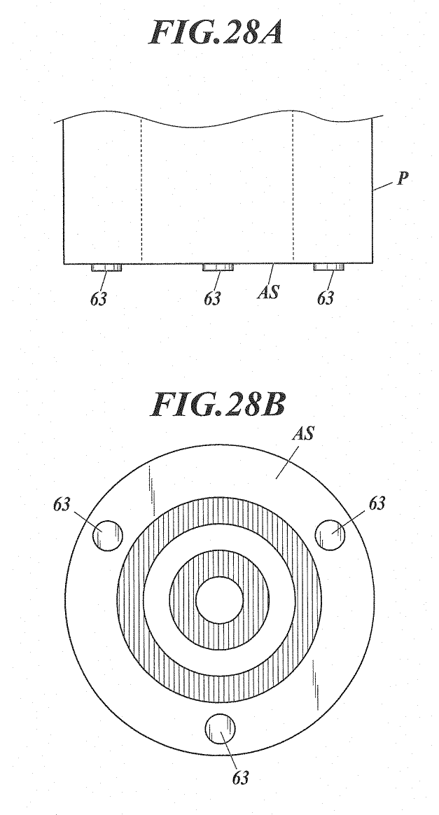

Additional sensors different from those of the measurement system of the probe P are disposed to directly or indirectly detect the tilt of the probe P or the unevenness of the contact of the end face AS with the surface BS. As illustrated in FIG. 28A and FIG. 28B, sensors 63 are disposed on the end face AS. The sensors 63 may be of any type, for example, a pressure sensor capable of detecting the tilt of the probe P or the unevenness of the contact of the end face AS with the surface BS through the pressure distribution or a contact sensor capable of detecting the imperfect contact.

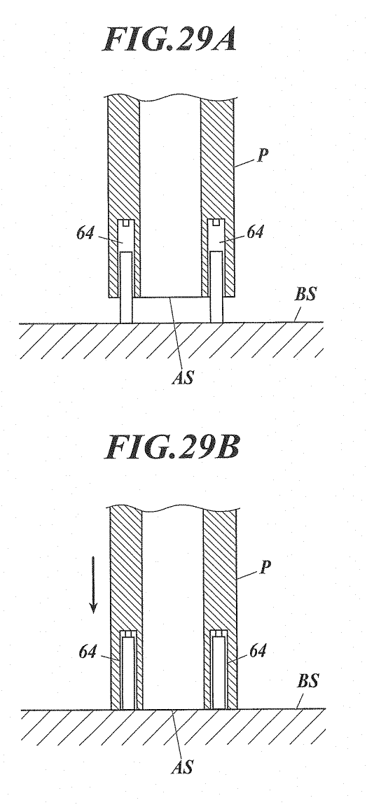

As illustrated in FIG. 29A and FIG. 29B, mechanical sensors of any type may also be provided at the end face AS, in addition to the sensors in the measuring system. For example, movable or stroking switches 64 extending from the end face AS of the probe P is provided. The switches 64 have different momenta (forces for returning from the ON-state to the OFF-state). When the probe P is urged onto the surface BS, one of the switches 64 is not put into ON-state. Pressing of all the switches 64 starts the measurement of the intensity of scattered light. Thus, the end face AS is kept in close contact with the surface BS during the measurement.

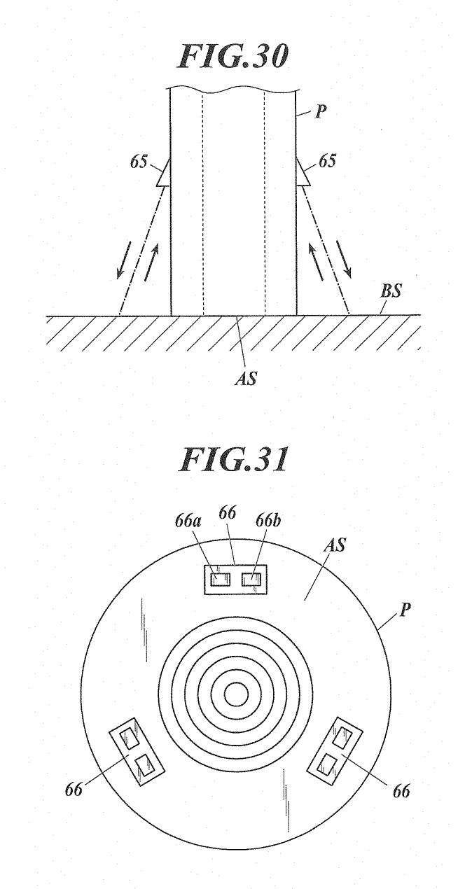

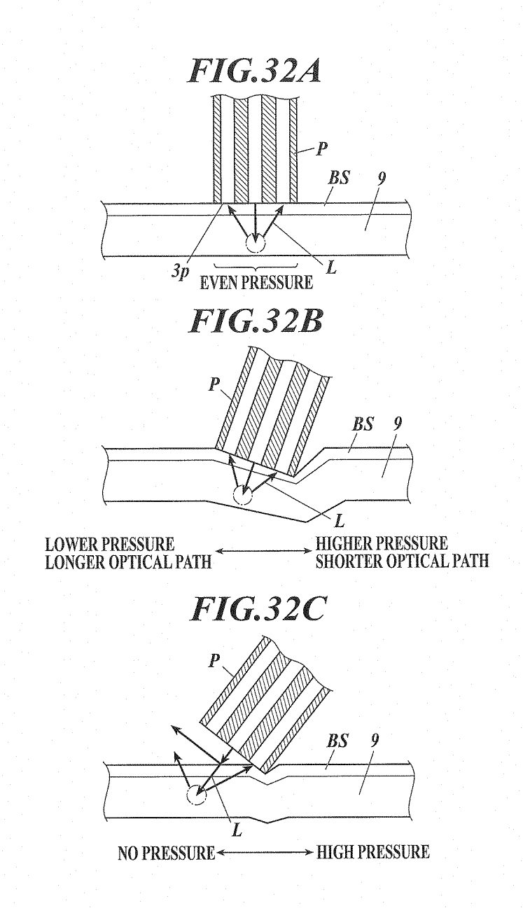

As illustrated in FIG. 30, a plurality of range sensors 65 may be disposed on the probe P to determine the distance to the surface BS. The range sensors 65 may be of any type, for example, a laser range sensor. For example, three or more range sensors 65 are disposed on the outer circumferential face of the probe at a predetermined angle. This allows the measurement of the angle of the probe P relative to the surface BS.

As illustrated in FIG. 31, reflective photodetectors 66 may be circumferentially disposed at the end face AS of the probe P, in addition to the sensors of the probe. The reflective photodetectors 66 each include light emitting devices 66a and 66b. The photodetectors 66 receive reflected scattered light to determine the distances between any site on the end face AS and the surface BS or the unevenness of the contact of the end face AS with the surface BS.

The wavelengths of the light beams to be emitted by the light emitting devices 66a may be equal to the wavelength of the light beam to be detected from the probe P.

The detailed configurations or operations of the components of the light radiation/reception system and the optical biometer according to the embodiments can be appropriately varied without departing from the scope and spirit of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-074710, filed on 9 Apr. 2018, is incorporated herein by reference in its entirety.

What is claimed is:

1. A light emission and reception system for optical biometric measurement, comprising:
a light radiator that radiates source light to a living body; and
a light receiver that receives return light scattered from the living body,
wherein
the light radiator and the light receiver are disposed at an end face of the system which is to be put into contact with a surface of the living body during the optical biometric measurement,
the system measures biometric data of the living body based on intensity of the light detected by the light receiver, and
(i) the light receiver comprises segments which respectively detect an intensity of the return light and which are at different angular ranges around a center axis of the light radiator, and/or (ii) the light radiator comprises segments which respectively radiate the source light and which are at different angular ranges around a center axis of the light receiver.

2. The system according claim 1, further comprising:
a backward optical fiber bundle that includes an inlet and an outlet; and
a light detecting component,
wherein
the backward optical fiber bundle guides the return light scattered from the living body through the inlet to the outlet toward a light detection side,
the light receiver is constituted by the inlet of the backward optical fiber bundle,
the outlet of the backward optical fiber bundle comprises segments that correspond to the segments of the light receiver, and
the light detecting component detects an intensity of the light from the segments of the outlet of the backward optical fiber bundle.

3. The system according claim 2, wherein the angular ranges of the segments of the light receiver in the inlet of the backward optical fiber bundle at the end face is:
(i) maintained;
(ii) converted into distribution on a specific coordinate; or
(iii) converted at random in each of the segments at the outlet of the backward optical fiber bundle.

4. The system according claim 2, wherein the light detecting component is photodetectors that respectively correspond to the segments of the outlet of the backward optical fiber bundle.

5. The system according claim 2, wherein the light detecting component is a two-dimensional image sensor that extends over the segments of the outlet of the backward optical fiber bundle.

6. The system according claim 1, wherein the light receiver further comprises photodetectors which are at the end face and which correspond to the segments of the light receiver.

7. The system according claim 1, wherein the light receiver is constituted by a two-dimensional image sensor at the end face.

8. The system according claim 1, further comprising:
a forward optical fiber bundle that includes an inlet and an outlet,
wherein
the forward optical fiber bundle guides the source light from a light source through the inlet to the outlet toward the living body, and
the light radiator is constituted by the outlet of the forward optical fiber bundle.

9. The system according claim 1, wherein the light radiator is constituted by light emitting elements at the end face.

10. The system according claim 1, wherein the segments of the light receiver as light receiving areas are disposed at substantially equal intervals around the center axis of the light radiator.

11. The system according claim 1, wherein a number of the segments of the light receiver is three or more.

12. The system according claim 1, wherein the light receiver that comprises the segments are (i) solid circular, square or rectangular, or (ii) hollow circular, square or rectangular.

13. The system according claim 1, wherein the segments of the light receiver are separately disposed around the center axis of the light radiator.

14. The system according claim 2, wherein the segments of the outlet of the backward optical fiber bundle are horizontally disposed.

15. The system according claim 2, wherein the segments of the outlet of the backward optical fiber bundle are circumferentially disposed.

16. The system according claim 1, further comprising:
a forward optical fiber bundle that includes an inlet and an outlet; and
a light source,
wherein
the forward optical fiber bundle guides the source light from the light source through the inlet to the outlet toward the living body,
the light radiator is constituted by the outlet of the forward optical fiber bundle,
the inlet of the forward optical fiber bundle comprises segments that correspond to the segments of the light radiator, and
the light source emits light toward the segments of the inlet of the forward optical fiber bundle.

17. The system according claim 16, wherein the angular ranges of the segments of the light radiator in the outlet of the forward optical fiber bundle at the end face is:
(i) maintained;
(ii) converted into distribution on a specific coordinate; or (iii) converted at random in each of the segments at the inlet of the forward optical fiber bundle.

18. The system according to claim 16, wherein the light source is light emitting elements that respectively correspond to the segments of the inlet of the forward optical fiber bundle.

19. The system according to claim 16, wherein the light source is a two-dimensional pixel matrix that extends over the segments of the forward optical fiber bundle.

20. The system according claim 1, wherein the light radiator is constituted by light emitting elements which are at the end face and which respectively correspond to the segments of the light radiator.

21. The system according claim 1, wherein the light radiator is constituted by a display with a two-dimensional pixel matrix at the end face.

22. The system according claim 1, further comprising:
a backward optical fiber bundle that includes an inlet and an outlet,
wherein
the backward optical fiber bundle guides the return light scattered from the living body through the inlet to the outlet toward a light detection side, and
the light receiver is constituted by the inlet of the backward optical fiber bundle.

23. The system according claim 1, wherein the light receiver is constituted by photodetectors at the end face.

24. The system according claim 1, wherein the segments of the light radiator as light radiating areas are disposed at substantially equal intervals around the center axis of the light receiver.

25. The system according claim 1, wherein a number of the segments of the light radiator is three or more.

26. The system according claim 1, wherein the light radiator that comprises the segments are (i) solid circular, square or rectangular, or (ii) hollow circular, square or rectangular.

27. The system according claim 1, wherein the segments of the light radiator are separately disposed around the center axis of the light receiver.

28. The system according claim 16, wherein the segments of the inlet of the forward optical fiber bundle are horizontally disposed.

29. The system according claim 16, wherein the segments of the inlet of the forward optical fiber bundle are circumferentially disposed.

30. The system according claim 1, wherein illumination light emitters are disposed near the light radiator and the light receiver at the end face.

31. An optical biometer comprising:
the light emission and reception system according claim 1; and
a control/arithmetic processor that controls radiation of the source light through the light radiator and calculates an observed result from the return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
if a difference among the light intensities detected for the segments of the light radiator falls within a predetermined threshold, the control/arithmetic processor calculates the observed result.

32. The biometer according to claim 31, wherein, if the difference among the light intensities detected for the segments of the light radiator does not fall within the predetermined threshold, the control/arithmetic processor controls re-radiation of the source light through the light radiator.

33. An optical biometer comprising:
the light emission and reception system according claim 1; and
a control/arithmetic processor that controls radiation of the source light through the light radiator and calculates an observed result from a return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
the control/arithmetic processor controls preliminary radiation of the source light and, if a difference among the light intensities detected for the segments of the light radiator during the preliminary radiation falls within a predetermined threshold, the control/arithmetic processor controls true radiation of the source light and calculates the observed result from true light intensities detected in the light receiver.

34. An optical biometer comprising:
the light emission and reception system according claim 1; and
a control/arithmetic processor that controls radiation of the source light through the light radiator and calculates an observed result from a return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
the control/arithmetic processor controls the light radiator not to radiate and, if a difference among light intensities detected for the segments of the light radiator during no radiation falls within the predetermined threshold, the control/arithmetic processor controls true radiation of the source light and calculates the observed result from the true light intensities detected in the light receiver.

35. An optical biometer comprising:
the light emission and reception system according claim 1; and
a control/arithmetic processor that controls radiation of the source light through the light radiator and calculates an observed result from a return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
if a difference among the light intensities detected for the segments of the light radiator exceeds a predetermined threshold, the control/arithmetic processor calculates the observed result from the light intensities detected for some of the segments of the light radiator.

36. The biometer according to claim 35, further comprising:
a display,
wherein the control/arithmetic processor instructs the display to indicate a low reliability of the observed result due to the difference among the light intensities exceeding the predetermined threshold.

37. An optical biometer comprising:
the light emission and reception system according claim 1; and
a control/arithmetic processor that controls radiation of the source light through the light radiator and calculates an observed result from a return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
the control/arithmetic processor (i) controls predetermined cycles of sequential radiation of the source light for each of the segments of the light radiator, (ii) calculates a difference among the light intensities for the segments for each cycle of radiation, and (iii) calculates the observed result from relatively even light intensities detected in the light receiver.

38. The biometer according to claim 31, further comprising:
a display,
wherein the control/arithmetic processor instructs the display to indicate that the difference among light intensities detected in the light receiver for the segments of the light radiator exceeds the predetermined threshold.

39. The biometer according to claim 31, further comprising:
a display,
wherein the control/arithmetic processor instructs the display to indicate (i) a direction of tilt of the end face against the surface of the living body, or (ii) a direction for correction to reduce the tilt.

40. The biometer according to claim 31, further comprising:
a display,
wherein
the control/arithmetic processor calculates a first observed result from the difference among the light intensities that falls within the predetermined threshold and instructs the display to present the first observed result,
the control/arithmetic processor calculates a second observed result from the difference among the light intensities that exceeds the predetermined threshold and instructs the display to present the second observed result, and
the control/arithmetic processor instructs the display to distinctively present the first observed result and the second observed result.

41. An optical biometer comprising:
the light emission and reception system according claim 1; and
a control/arithmetic processor that controls radiation of the source light through the light radiator and calculates an observed result from a return light detected in the light receiver,
wherein
if the light receiver comprises one segment and the light radiator comprises multiple segments, the control/arithmetic processor controls time-divisional radiation of the source light from the segments of the light radiator and detection of the sequential return lights in the segment of the light receiver to obtain light intensities for the segments of the light radiator, and
in order to calculate the observed result from the light intensities for the segments of the light radiator, the control/arithmetic processor adds an offset according to the difference among the light intensities for the segments.
